(19) United States
(12) Patent Application Publication
(10) Pub. No.: US 2010/0080783 A1
(43) Pub. Date: Apr. 1, 2010

(54) Microbial feed additive

(76) Inventor: James B. Watson, Pierce, NE (US)

Correspondence Address: THOMTE LAW OFFICE, L.L.C., 2120 S. 72ND STREET, SUITE 1111, OMAHA, NE, 68124, US

(21) Appl. No.: 12/592,590

(22) Filed: Nov. 30, 2009

Related U.S. Application Data
(62) Division of application No. 10/994,949, filed on Nov. 22, 2004.

(51) Int. Cl. A61K 35/74; A61P 43/00

(52) U.S. Cl. 424/93.45

(57) ABSTRACT

A microbial feed additive comprising lactic acid bacteria, silicate such as bentonite, and a yeast culture. The animal feed additive of this invention results in more piglets born alive, reduced mortality in nursery pigs, improved growth performance in pregrower, growing and finishing pigs, improved utilization of dietary protein in pregrower and finishing pigs, and improved growth performance in stressed pigs.

### CROSS-REFERENCE TO RELATED APPLICATION

This is a divisional application of Applicant's earlier application Ser. No. 10/994,949 filed Nov. 22, 2004, entitled MICROBIAL FEED ADDITIVE.

### BACKGROUND OF THE INVENTION

1. Field of the Invention

This invention relates to a microbial feed additive for animal feeds such as swine, chickens, turkeys, etc.

2. Description of the Related Art

Many types of animal feed additives have been utilized for many years to enhance animal growth, to reduce mortality rates in nursery pigs, etc. For the past two years, the inventor, with the assistance of the assignee of this invention, has developed a microbial feed additive for the swine industry. Although the field research has been conducted with pigs, applicant anticipates that the feed additive of this invention may be extended to include chickens and turkeys (monogastrics). The product of this invention consists of three main components: lactic acid bacteria (LAB), silicate such as bentonite, and yeast.

### SUMMARY OF THE INVENTION

The microbial feed additive of this invention comprises lactic acid bacteria (LAB), silicate such as bentonite, and yeast. In the preferred embodiment, the lactic acid bacteria is selected from the group of Bacillus subtilis, Bifidobacterium adolescentis, Bifidobacterium animalis, Bifidobacterium bifudum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidlactici, Pediococcus pentosaceus, Streptococcus cremoris, Streptococcus discetylactis, Streptococcus faecium, Streptococcus intermedius, Streptococcus lactis, and Streptococcus thermophilus. The lactic acid bacteria is added to the feed at the rate of 100 to 100,000,000 cfu/g, the silicate comprises 0.0005 to 5.0% of the total mixed ration and the yeast culture comprises 0.000025 to 2.5% of the total mixed ration. In the preferred feed additive, the LAB is added to the feed at the rate of 130,000 cfu/g while the bentonite comprises 0.05% of the total mixed ration and the yeast culture comprises 0.0025% of the total mixed ration.

The results of feeding trials conducted to date substantiates that the microbial feed additive of this invention increases the percentage of piglets born alive approximately 5.9%, improves the mortality rate of the number of pigs in the amount of 59.1%, and increases growth performance, i.e., gain, in substantial amounts as will be set forth in the tables reproduced hereinbelow.

It is therefore a principal object of the invention to provide an improved animal feed additive.

A further object of the invention is to provide an improved microbial feed additive.

Yet another object of the invention is to provide a microbial feed additive consisting of three main components, namely, lactic acid bacteria, a silicate such as bentonite, and yeast.

Still another object of the invention is to provide a microbial feed additive which increases growth performance.

Still another object of the invention is to provide a microbial feed additive which increases the number of piglets born alive.

Still another object of the invention is to provide a microbial feed additive which improves the mortality rate of pigs.

These and other objects will be apparent to those skilled in the art.

### DETAILED DESCRIPTION OF THE PREFERRED MICROBIAL FEED ADDITIVE

For the past two years, Applicant, with the assistance of the assignee of this invention, has developed a microbial feed additive for the swine industry. Although the field research has been conducted with pigs, applicant anticipates that the additive of this invention may be extended to include chickens and turkeys (monogastrics). The feed additive of this invention comprises three main components, lactic acid bacteria (LAB), silicate such as bentonite, calcium bentonite, sodium bentonite, perlite, verxite, zeolite montmorillonite, and yeast. The yeast of this invention may be either a live or a dead yeast. In the animal feed additive of this invention, the lactic acid bacteria is selected from the group of Bacillus subtilis, Bifidobacterium adolescentis, Bifidobacterium animalis, Bifidobacterium bifudum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidlactici, Pediococcus pentosaceus, Streptococcus cremoris, Streptococcus discetylactis, Streptococcus faecium, Streptococcus intermedius, Streptococcus lactis, and Streptococcus thermophilus. The amount of lactic acid bacteria added to the animal feed ration ranges in the amount of 100 to 100,000,000 cfu/g, the silicate comprises 0.0005 to 5.0% of the total mixed ration, while the yeast comprises 0.0025 to 2.5% of the total mixed ration. In the preferred formula, the lactic acid bacteria is added to the animal feed in the amount of 130,000 cfu/g, the silicate comprises 0.05% of the total mixed ration and the yeast culture comprises 0.0025% of the total mixed ration.

The silicate of the animal feed additive of this invention may be bentonite, calcium bentonite, sodium bentonite, perlite, verxite, or zeolite. Bentonite is a naturally occurring mineral consisting primarily of the tri-layered aluminum silicate, montmorillonite. It may contain calcium or sodium as the predominant available or exchange ion. Bentonite is normally used or intended for use in non-medicated animal feed as an anti-caking agent or a pelleting aid. Although bentonite was used in the trials set forth below, it is believed that sodium bentonite or calcium bentonite are also satisfactory in the feed additive of this invention.

Hereinbelow are Tables 1-7 which set forth the results of the exhaustive tests conducted by applicant and the assignee wherein the feed additive of this invention is identified as Formula 1050.

TABLE 1
Effect of feeding Formula 1050 on piglets born alive per sow.

| Item | Formula 1050 | No Formula 1050 |
|---|---|---|
| # Sows farrowed | 505 | 625 |
| # Piglets born alive | 12.5 | 11.8 |
| Improvement | 5.9% | |

TABLE 2
Effect of feeding Formula 1050 on growth performance and
mortality in Nursery Pigs (34 d).

| Item | Formula 1050 | No Formula 1050 |
|---|---|---|
| # Pigs | 6391 | 6129 |
| Gain per pig, lb | 40.3 | 39.3 |
| Improvement | 2.5% | |
| Mortality, # pigs | 65 | 159 |
| Improvement | 59.1% | |

TABLE 3
Effect of feeding Formula 1050 with reduced dietary protein on growth
performance in Growing-Finishing pigs (70 d).

| | Formula 1050 | No Formula 1050 |
|---|---|---|
| Item | reduced soybean meal | regular soybean meal |
| # Pigs | 5831 | 5784 |
| Gain per pig, lb | 114.1 | 98.4 |
| Improvement | 15.9% | |

TABLE 4
Effect of feeding Formula 1050 on growth performance in “Pregrower”
barrows fed diets containing Salinomycin (27 d).

| | Formula 1050 | No Formula 1050 |
|---|---|---|
| Item | reduced soybean meal | regular soybean meal |
| # Barrows | 359 | 433 |
| Gain per pig, lb | 45.7 | 42.5 |
| Improvement | 7.5% | |

TABLE 5
Effect of feeding Formula 1050 on growth performance in “Pregrower”
gilts fed diets containing Salinomycin (34 d).

| | Formula 1050 | No Formula 1050 |
|---|---|---|
| Item | reduced soybean meal | regular soybean meal |
| # Gilts | 328 | 433 |
| Gain per pig, lb | 55.9 | 52.4 |
| Improvement | 6.7% | |

TABLE 6
Effect of feeding “Lincomix + Superzyme” or Formula 1050
on growth performance of “Growing pigs”.

| | Formula | Lincomix + | No Medication + |
|---|---|---|---|
| Item | 1050 | Superzyme | No Formula 1050 |
| Daily feed, lb | 5.90 | 6.03 | 6.14 |
| Improvement vs Control | 3.9% | 1.8% | |
| Daily gain, lb | 2.10 | 2.12 | 1.81 |
| Improvement vs Control | 16% | 17.1% | |
| Feed/gain | 2.91 | 2.92 | 3.45 |
| Improvement vs Control | 15.6% | 15.4% | |

TABLE 7
Effect of feeding Formula 1050 on growth performance of “Finishing”
pigs.

| | Formula 1050 | No Formula 1050 |
|---|---|---|
| Item | reduced soybean meal | regular soybean meal |
| Daily feed, lb | 5.03 | 5.97 |
| Improvement | 15.7% | |
| Daily gain, lb | 2.02 | 2.04 |
| Improvement | −1.0% | |
| Feed/gain | 2.49 | 2.94 |
| Improvement | 15.3% | |

As seen in Table 1, the use of the feed additive of this invention results in an improvement in the number of piglets born alive in the amount of 5.9%.

Table 2 illustrates that the use of the feed additive of this invention results in an improvement in the gain per pig in the amount of 2.5% and results in a reduction in the mortality rate of the pigs in the amount of 59.1%.

Table 3 illustrates that the effect of feeding the animal feed additive of this invention with reduced dietary protein on growth performance in growing-finishing pigs increases the gain per pig in the amount of 15.9%.

Table 4 illustrates an improved gain per pig in the amount of 7.5% while Table 5 illustrates an improved gain per pig in the amount of 6.7%.

Table 6 illustrates the effect of feeding Lincomix+Superzyme on growth performance of growing pigs. It can be seen from Table 6 that the animal feed additive of this invention provides a dramatic improvement.

Table 7 also illustrates the results of the effect of feeding the feed additive of this invention with respect to finishing pigs.

It is clear from the above tables that the use of the feed additive of this invention in a feed ration results in: (1) more piglets born alive; (2) reduced mortality in “nursery” pigs; (3) improved growth performance in “pregrower”, “growing” and “finishing” pigs; (4) improved utilization of dietary protein in “pregrower” and “finishing” pigs; and (5) improved growth performance in “stressed” pigs.

The additive utilized in the feeding trials of Tables 1-7 above consisted of the ingredients set forth in Table 8 hereinbelow. The amounts set forth in Table 8 are the amounts in the total mixed ration.

TABLE 8

| Ingredient | Amount in Total Mixed Ration |
|---|---|
| LAB, cfu/g | 130,000 |
| Bentonite % | 0.05 |
| Yeast culture % | 0.0025 |

While Table 8 sets forth the preferred amounts of the ingredients, Table 9 hereinbelow sets forth the permissible ranges of those ingredients.

TABLE 9

| Ingredient | Permissible Amount in Total Mixed Ration |
|---|---|
| LAB, cfu/g | 100-100,000,000 |
| Bentonite % | 0.0005-5.0 |
| Yeast culture % | .000025-2.5 |

It can therefore be seen that the microbial feed additive of this invention represents a significant improvement in the art. Thus it can be seen that the feed additive of this invention accomplishes at least all of its stated objectives.

1. An animal feed additive, said animal feed additive comprising lactic acid bacteria, yeast and silicate.

2. The animal feed additive of claim 1 wherein said lactic acid bacteria is selected from the group consisting of Bacillus subtilis, Bifidobacterium adolescentis, Bifidobacterium animalis, Bifidobacterium bifudum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidlactici, Pediococcus pentosaceus, Streptococcus cremoris, Streptococcus discetylactis, Streptococcus faecium, Streptococcus intermedius, Streptococcus lactis, and Streptococcus thermophilus.

3. The animal feed additive of claim 1 wherein said yeast comprises live yeast.

4. The animal feed additive of claim 1 wherein said yeast comprises dead yeast.

5. The animal feed additive of claim 1 wherein said silicate comprises bentonite.

6. The animal feed additive of claim 1 wherein said silicate comprises calcium bentonite.

7. The animal feed additive of claim 1 wherein said silicate comprises sodium bentonite.

8. The animal feed additive of claim 1 wherein said silicate comprises perlite.

9. The animal feed additive of claim 1 wherein said silicate comprises verxite.

10. The animal feed additive of claim 1 wherein said silicate comprises zeolite.

11. The animal feed additive of claim 1 wherein said silicate comprises tri-layered aluminum silicate.

12. The animal feed additive of claim 1 wherein said silicate comprises montmorillonite.
